(19) United States
(12) Patent Application Publication
(10) Pub. No.: US 2018/0296394 A1
(43) Pub. Date: Oct. 18, 2018

(54) WOUND DRESSING WITH ELASTIC PROPERTIES FOR CONTOURING TO THE SHAPE OF A LIMB

(71) Applicant: ALESSANDRO BARBERIO, Aurora (CA)

(72) Inventor: ALESSANDRO BARBERIO, Aurora (CA)

(21) Appl. No.: 15/766,126

(22) PCT Filed: Oct. 5, 2016
(86) PCT No.: PCT/CA2016/051157

§ 371 Date: Apr. 5, 2018

Related U.S. Application Data
(60) Provisional application No. 62/284,602, filed on Oct. 5, 2015.
(60) Provisional application No. 62/387,615, filed on Jan. 7, 2016.
(60) Provisional application No. 62/389,341, filed on Feb. 24, 2016.
(60) Provisional application No. 62/496,007, filed on Oct. 3, 2016.

(51) Int. Cl. A61F 13/00; A61F 13/04; A61F 5/058

(52) U.S. Cl. CPC A61F 13/00038; A61F 13/00029; A61F 13/046; A61F 2013/00272; A61F 2013/00119; A61F 2013/00293; A61F 2013/00255; A61F 5/058

(57) ABSTRACT

In accordance with an aspect of the present invention, there is provided a wound dressing with elastic properties for use with a human or an animal. The wound dressing includes a plurality of individual segments, each segment connected to at least one other segment by an elastic connector, and each segment having at least two perforated layers, with at least one layer having a plurality of protrusions. The wound dressing further includes a plurality of spacing elements, with each spacing element disposed between adjacent segments and retained by either the adjacent segments or the elastic connector.

### FIELD OF THE INVENTION

The present specification relates generally to wound dressings and more specifically relates to a wound dressing with elastic properties for use with orthopedic devices such as casts and walking braces.

### BACKGROUND OF THE INVENTION

Wound dressings are used in the treatment of wounds for humans and animals. Wound dressings can aid in the treatment of a wound by, for example, providing aeration via passage of oxygen to a wound, protecting a wound against external shocks, storing medication for the healing of a wound, and storing exudate from a wound. Wound dressings may be applied directly to wound or may be applied to the internal wall of an orthopedic device such as a cast or a brace.

Currently, wound dressings are required to be manufactured in a large variety of shapes and sizes in order to accommodate the various sizes and types of wounds and limbs bearing wounds. Additionally, various orthopedic devices are custom-made in the event an existing orthopedic device does not fit the limb bearing the wound that requires treatment. This plethora of orthopedic devices to accommodate the countless wound variations is inefficient, time-consuming and expensive.

In particular, issues with wound dressing and orthopedic devices arise when dealing with limbs having excessive and variable contours, such as the calves or biceps, as well as when dealing with variation in contours created by muscle flexion and extension while the limb is in use.

Accordingly, there remains a need for improvements in the art.

### SUMMARY OF THE INVENTION

In accordance with an aspect of the invention, there is provided a wound dressing including at least two layers for application on a patient's wound. The patient may be a human or an animal. The wound dressing may comprise a plurality of individual segments, each segment connected to at least one other segment by an elastic connector, each segment comprising at least two perforated layers, wherein at least one layer comprises a plurality of protrusions; and a plurality of spacing elements, each spacing element disposed between adjacent segments and retained by either the adjacent segments or the elastic connector.

Other aspects and features according to the present application will become apparent to those ordinarily skilled in the art upon review of the following description of embodiments of the invention in conjunction with the accompanying figures.

### BRIEF DESCRIPTION OF THE DRAWINGS

Reference will now be made to the accompanying drawings which show, by way of example only, embodiments of the invention, and how they may be carried into effect, and in which:

FIG. 1 is a view of a wound dressing according to an embodiment of the invention;

FIG. 2 is a view of a wound dressing according to an embodiment of the invention;

FIG. 3 is a view of a wound dressing according to an embodiment of the invention;

FIG. 4 is a view of a wound dressing according to an embodiment of the invention;

FIG. 5a is a view of a disassembled wound dressing according to an embodiment of the invention;

FIG. 5b is a view of the assembled dressing of FIG. 5a;

FIG. 6 is a view of a segment of a wound dressing according to an embodiment of the invention;

FIG. 7A is a view of a segment of a wound dressing according to an embodiment of the invention;

FIG. 7B is a magnified view of apportion of FIG. 7A;

FIG. 8 is a view of an unassembled wound dressing according to an embodiment of the invention;

FIG. 9 is a view of the assembled wound dressing of FIG. 8;

FIGS. 10 to 16 are views of variations of shape for the wound dressing of FIG. 8;

FIG. 17 is a planar view of a sheet of a wound dressing according to an embodiment of the invention;

FIG. 18 is a perspective view of a wound dressing according to an embodiment of the invention;

FIGS. 19 and 20 are views of a protruded layer in accordance with an embodiment of the invention;

FIGS. 21 to 23 are views of a protruded layer in accordance with an embodiment of the invention;

FIG. 24 is a view of a wound dressing applied to a wound in accordance with an embodiment of the invention;

FIG. 25 is a view of a wound dressing according to an embodiment of the invention;

FIG. 26 is a view of a wound dressing according to an embodiment of the invention;

FIG. 27 is a view of the dressing of FIG. 26 including segments and “H” channels;

FIG. 28 is a view of a wound dressing according to an embodiment of the invention wrapped around a human calf and foot;

FIG. 29A-C show a flexible material spacer wrapped in a tubinette in accordance with an embodiment of the invention; and

FIG. 30 is a view of a wound dressing using fabric spacers in accordance with an embodiment of the invention.

Like reference numerals indicate like or corresponding elements in the drawings.

### DETAILED DESCRIPTION OF THE EMBODIMENTS

The present invention relates to a wound dressing which may be used to cover and protect wounds on humans and animals, particularly to limbs and digits where wrapping the wound may require expansion of the dressing to accommodate varying shapes and dimensions of muscle and bone inherent to the limb, as well as to accommodate changes as a result of movement. The wound dressing is designed for use with orthopedic devices, such as casts and walking braces, but may also be used in isolation.

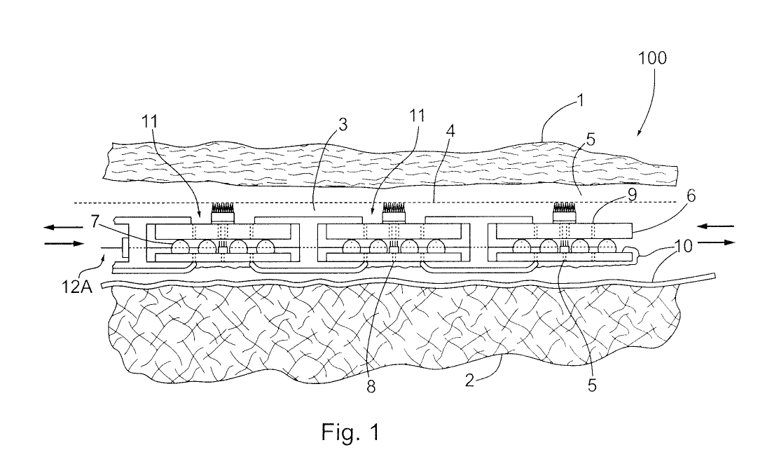

According to an embodiment as shown in FIG. 1, a wound dressing 100 may be located between a fiberglass or similar cast 1 and the patient's skin 2. The dressing 100 may be formed from segments 11 connected via expandable connector or belt, such as elastic net 4 or a four-way stretchable stokinette and separated from each other by spacing elements such as “H” channels 3. Soft fasteners 5, such as Velcro™ may be used to connected segments 11 to elastic net 4. Each segment 11 may be formed from a perforated layer 6 and a protruded layer 7, with perforations located between the protrusions and with holes 8 and 9 incorporated for ventilation. As shown, protruded layer 7 is located closest to the patient's skin 2, however, the layers may also be located in reverse order. “H” channels 3, perforated layer 6 and protruded layer 7 may be constructed from hypoallergenic materials such as rubber, silicone, plastic, foam, EVA, gel or any other suitable material known to a skilled person that could come into direct contact with a user's open wound. Elastic net 4 may be constructed of an elastic tubular dressing material such as Surgilast™ Segments 11 and “H” channels 3 may be enveloped with a perforated or porous cushion 10, which may be constructed of cotton or gel. Additionally, perforated layer 6 and protruded layer 7 may be attached to each other with soft fasteners 5, as described above, or by ultrasonic welding or other means, as well as perforated layer 6 being attached to elastic net 4 with soft fasteners.

Further to the embodiment shown in FIG. 1, each segment 11 is capable of movement along the direction indicated by the arrows to enable expansion of dressing 100 for wrapping around wounds and limbs of differing dimensions, both in overall dimensions and within the limb itself, to account for variances caused by bone and muscle e.g. a human calf. Spacing elements such as “H” channels 3 may act both to constrain movement of segments 11 to a maximum dimension as well as to prevent the patient's skin from being trapped or pinched between segments 11 during movement. According to an embodiment, each segment 11 may be further connected by elastic thread 12A. Elastic thread 12A may run transversally through the walls of “H” channels 3 and may be tensioned to assist in securing dressing 100.

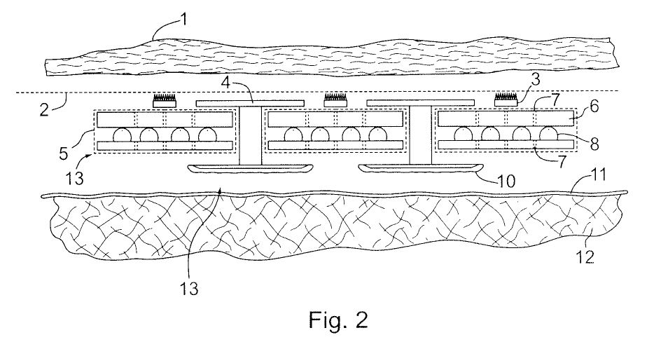

According to an embodiment as shown in FIG. 2, a wound dressing 100 may be located between a fiberglass or similar cast 1 and the patient's skin 12. The dressing 100 may be formed from segments 13 connected via “H” channels 4 and an elastic net 2. Soft fasteners 3, such as Velcro™, may be used to connected segments 13 to elastic net 2. Each segment 13 may be formed from a perforated layer 6 and a protruded layer 8, with protruded layer 8 closest to the patient's skin 12 (layers may be reversed, if desired according to an embodiment), and with holes 7 incorporated for ventilation. Each segment 13 is further wrapped in a tubinette 5 constructed of cotton such as Surgitube™ or Tubinette™. “H” channels 4, perforated layer 6 and protruded layer 8 may be constructed from hypoallergenic materials such as rubber, silicone, plastic, foam, EVA, gel or any other suitable material known to a skilled person that could come into direct contact with a user's open wound. Elastic net 2 may be constructed of an elastic tubular dressing material such as Surgilast™. “H” channels 4 may be enveloped with a perforated cushion 10, which may be constructed of cotton or gel. Additionally, perforated layer 6 may be attached to elastic net 2 with soft fasteners as described above.

Further to the embodiment shown in FIG. 2, each segment 13 is capable of movement along the direction indicated by the arrows to enable expansion of dressing 100 for wrapping around wounds and limbs. “H” channels 4 may act both to constrain movement as well as to prevent the patient's skin from being trapped or pinched between segments 13 during movement.

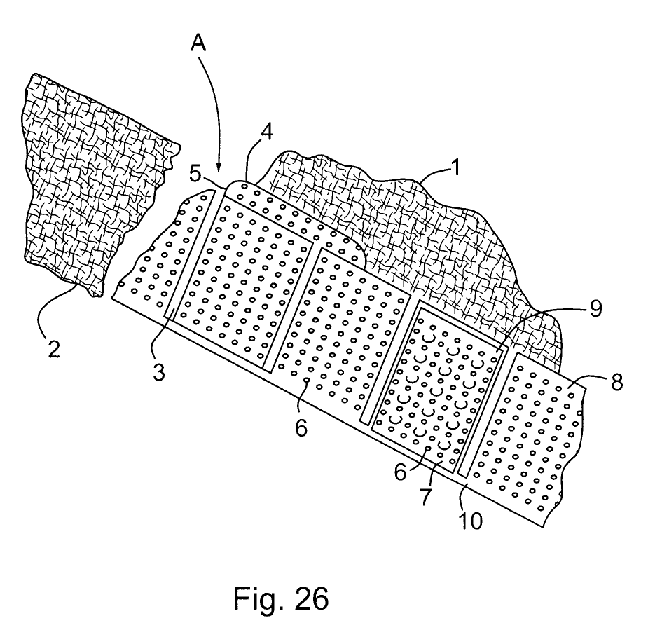

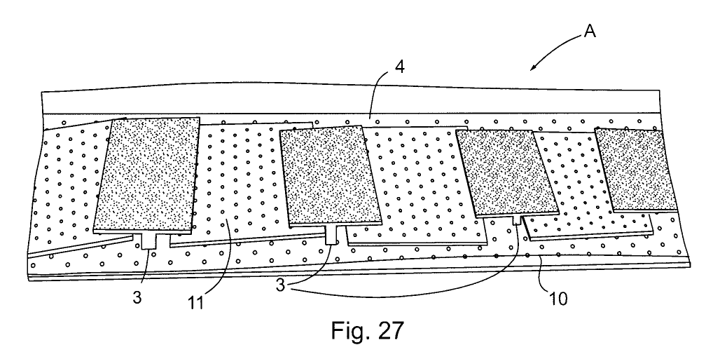

FIGS. 26 and 27 show an embodiment with multiple segments 11 arranged into a wound dressing A for use in treating wounds. The segments 11 are separated by spacers such as the “H” channels 3 passing through the through the expandable connector 10. Dressing A may then be wrapped around an injured limb, such as an arm or leg, and segments 11 may elasticize with expandable connector 10 to adjust to the contours of the limb, and then contract slightly when finished to assist in staying secure. “H” channels 3 enable the movement of the segments 11 within the “H” channel 3, as well as acting to prevent the skin or the wound from being pinched as the segments 11 extend and contract. The entire dressing A may be wrapped in a tubinette or similar stretchable material to form a unitary piece, which may then be dispensed and used as needed.

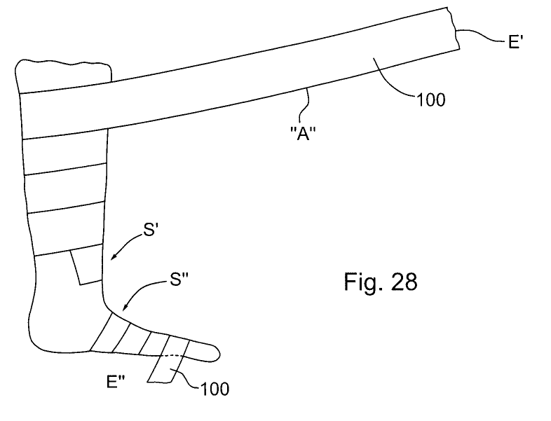

FIG. 28 shows wound dressing 100 as in FIG. 26 wrapped around a patient's lower leg. From the heel, one dressing “A” is wrapped from area S′ upwards along the calf towards the knee. A second dressing starts at S″ and wraps along the foot towards the toes at E″. A similar process may be followed for an arm injury, starting from the elbow, with one dressing running upwards from the elbow along the bicep, and a second dressing running from the elbow along the forearm to the hand and fingers. In either case, the wound dressing may then be covered with a cast or walking brace.

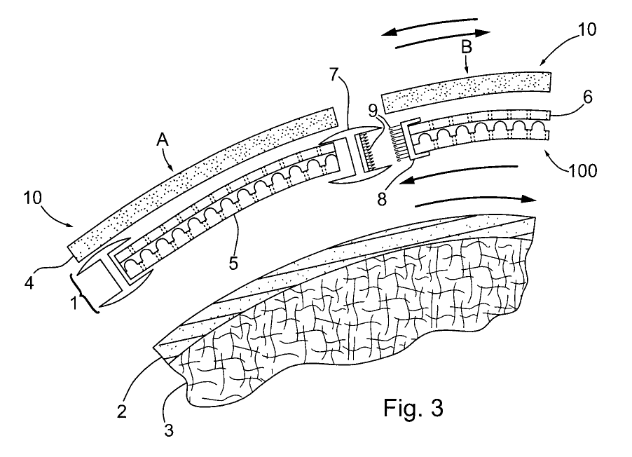

According to an embodiment as shown in FIG. 3, a wound dressing 100 may be located between a brace A and B for a limb and the patient's skin 3. The dressing 100 may be formed from segments 10 connected via “H” channels 7 and “U” channels 8 by a soft fastener 9, such as Velcro™ Each segment 10 may be formed from an external layer 4, a perforated layer 6 and a protruded layer 5, with protruded layer 5 closest to the patient's skin 2 (layers may be reversed, if desired), and with holes incorporated for ventilation. External layer 4 may be constructed from a rigid material, such as fiberglass or plastic, and “H” channels 7, “U” channels 8, perforated layer 6 and protruded layer 5 may be constructed from hypoallergenic materials such as rubber, silicone, plastic, foam, EVA or any other suitable material known to a skilled person that could come into direct contact with a user's open wound. Brace A may further include a wall 1 formed from a “H” channel where brace A acts as the edge of the dressing 100. A cushion 2 of cotton or gel may also be provided to protect the skin 3.

Further to the embodiment shown in FIG. 3, each segment 10 is capable of movement along the direction indicated by the arrows to enable brace A and B to be separated and reengaged for wrapping around wounds and limbs. “H” channels 7 and “U” channels 8 may act to both provide a point of engagement between segments 10 as well as to prevent the patient's skin from being trapped or pinched between segments 10 during movement. Further, by incorporating fastener 9, segments 10 may be better constrained with “H” channels 7, allow for greater bending or deformation of segments 10 if needed for application.

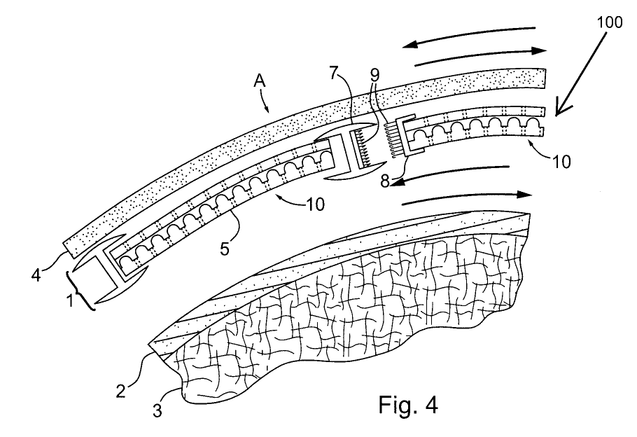

According to an embodiment as shown in FIG. 4, a wound dressing 100 may be located between a cast A for a limb and the patient's skin 3. The dressing 100 may be formed from segments 10 connected via “H” channels 7 and “U” channels 8 by a soft fastener 9, such as Velcro™. Each segment 11 may be formed from a perforated layer 6 and a protruded layer 5, with protruded layer 5 closest to the patient's skin 2 as shown (layers may be reversed, if desired), and with holes incorporated for ventilation. Cast A may be constructed from outer layer 4 of a rigid material, such as fiberglass or plastic, and “H” channels 7, “U” channels 8, perforated layer 6 and protruded layer 5 may be constructed from hypoallergenic materials such as rubber, silicone, plastic, foam, EVA or any other suitable material known to a skilled person that could come into direct contact with a user's open wound. Dressing 100 may further include a wall 1 formed from a “H” channel at the edge of cast A. A cushion 2 of cotton or gel may also be provided to protect the skin 3.

Further to the embodiment shown in FIG. 4, each segment 10 is capable of movement along the direction indicated by the arrows, as well as separating and reengaging to enable installation and positioning against rigid layer 4 prior to applying cast A to the patient. “H” channels 7 and “U” channels 8 may act to both provide a point of engagement between segments 10 as well as to prevent the patient's skin from being trapped or pinched between segments 10 during movement.

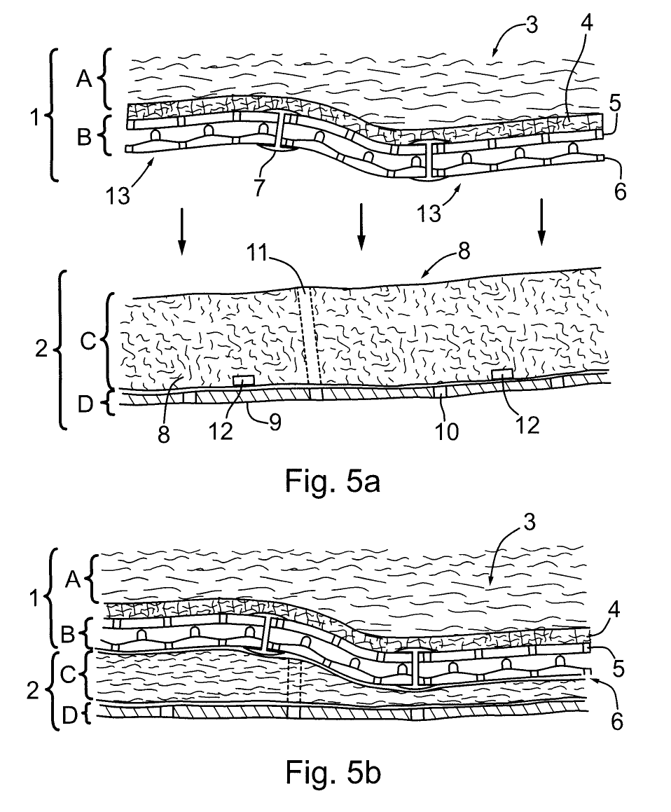

According to an embodiment as shown in FIG. 5a and FIG. 5b, a wound dressing B may be located between a cast 2 for a limb A and the patient's skin 3, with the combination of limb A and dressing B treated as a single body 1. The dressing B may be formed from segments 13 connected via “H” channels 7 to allow for adjustment, expansion and contraction. Each segment 11 may be formed from a perforated layer 5 and a protruded layer 6, with perforated layer 5 closest to the patient's skin 3, and with holes incorporated for ventilation. Cast 2 may be constructed from outer layer D of a rigid material, such as fiberglass or plastic, and a cushion layer C which is made of a compressible material, such as hypoallergenic foam. Outer layer D incorporates holes 10 to provide ventilation, as does cushion layer C via holes 11. Cushion layer C is secured to outer layer D via soft fasteners 12, such as Velcro™ Cushion layer C may further include external fabric layers 8.

As shown in FIG. 5b, when cast 2 is applied to body 1, cushion layer C is compressed as shown to ensure a tight fit around irregularities in the limb A, such a calf muscles, with dressing B providing ventilation in combination with holes 10 and 11.

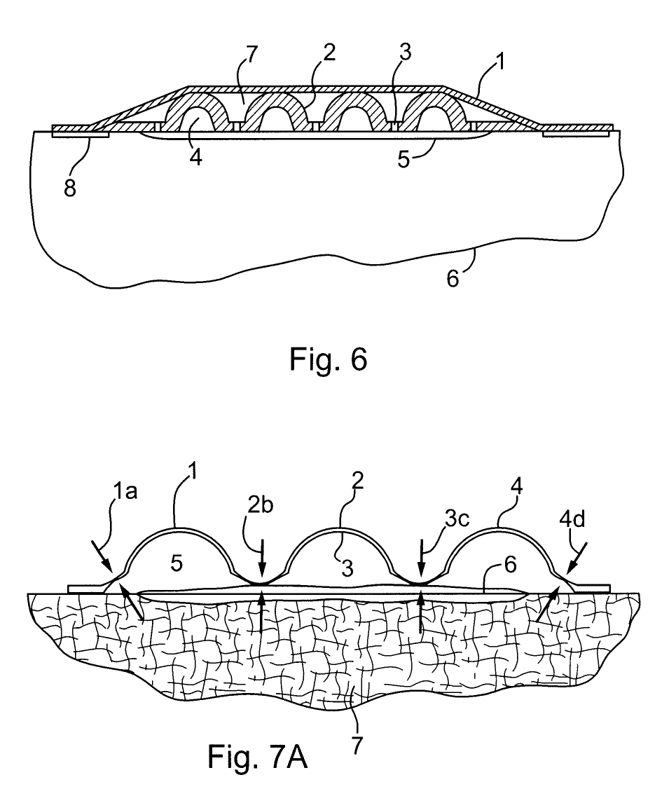

According to an embodiment as shown in FIG. 6, a segment for use in a wound dressing as described above may be formed from a wound cover film 1 and a protruded layer 2 which combine to overlay a wound 5 on a patient's skin 6. The segment may adhere to patient's skin 6 via adhesion layers 8 for use in isolation. The combination of wound cover film 1 with protruded layer 2 may result in cavities 4 and vented space 7 as part of the segment. Protruded layer 2 of the segment is closest to patient's skin 6, such that under each cavity 4 may act to store exudate from patient's wound 5. Protruded layer 2 may also include perforations 3 for ventilation into vented space 7. Wound cover film 1 may comprise holes 10 for ventilation. The segment may be at least twice as large in size than user's wound 5 for protection of the wound against external shocks. The protrusions of protruded layer 2 may vary in shape. Protrusions may be curved surfaces or comprise of cornered edges. Protrusions may allow absorption of exudate from user's wound 5 with less amount of substrate thus reducing the frequency of required changes of wound dressings.

According to an embodiment, perforations 3 may range from 100 to 1400 microns in diameter. Wound cover film 1 may range from 0.03 to 0.05 mm in thickness. Holes 10 may range from 100 to 1400 microns in diameter. Wound cover film 1 and protruded layer 2 may vary in overall thickness depending on the depth of patient's wound 5.

According to an embodiment as shown in FIG. 6, for example, wound cover film 1 may be 0.7 mm in thickness, protruded layer 2 may be 2 mm in thickness, perforations 3 may be 0.25 mm in diameter, each cavity 4 may be 1.6 mm in length, each protrusion of layer 2 may be 2 mm in length, and the distance between each protrusion of layer 2 may be 1.5 mm in length.

Wound cover film 1 may be constructed of polyurethane, plastic, fabric, textile, silicone, EVA, foam, Delnet™ or any other suitable material known to a skilled person but may not be constructed of latex. Wound cover film 1 may be clear, breathable, flexible, antibacterial or waterproof. Wound cover film 1 may comprise an aperture film, which provides precise rates of permeability. Protruded layer 2 may be constructed of Masterflow™ or Stratex™. Protruded layer 2 may comprise wicking properties, release and barrier characteristics, and defined absorbencies. Protruded layer 2 may be formed of up to five laminated layers, wherein there are no chemical adhesives or contaminants, and may be non-adhering and non-linting. According to an embodiment, protruded layer 2 may further be covered by a hard layer (not shown) made of EVA, polyurethane, transparent plastic or any other suitable material known to a skilled person.

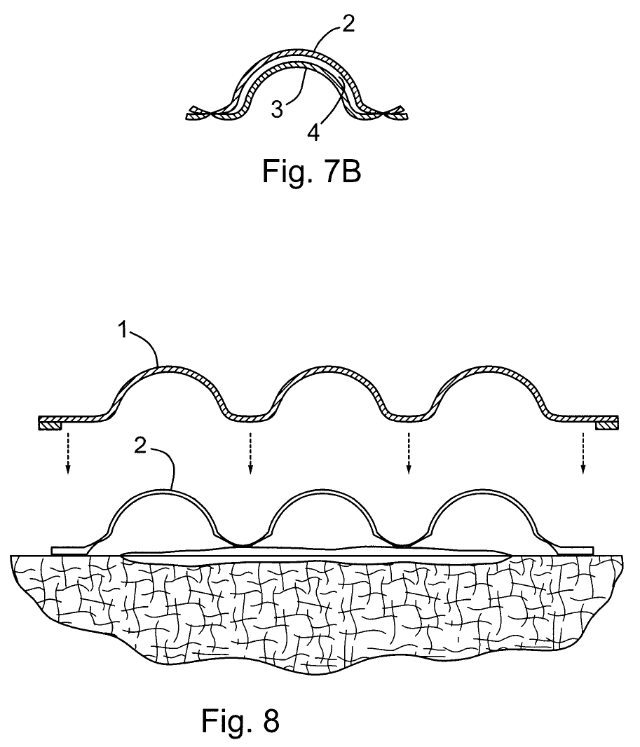

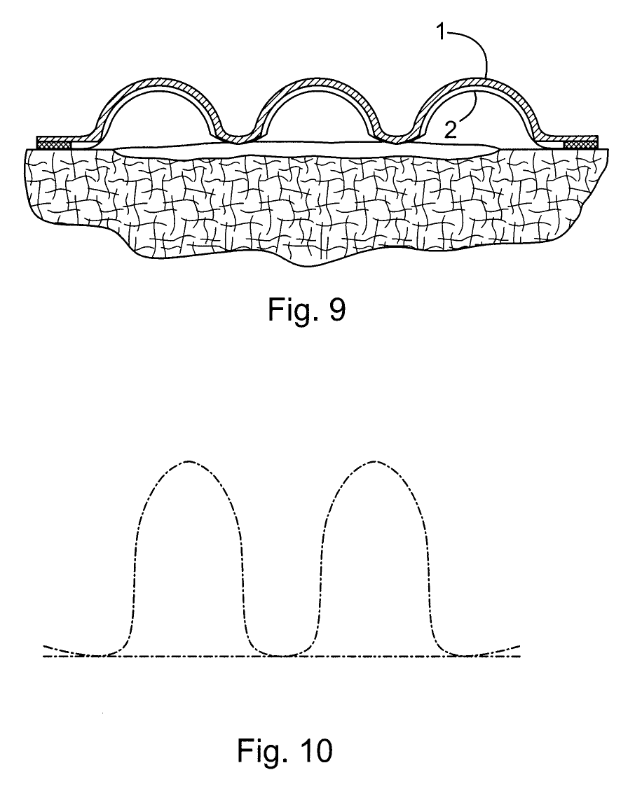

According to an embodiment as shown in FIG. 7A, and magnified in FIG. 7B, a segment for use in a wound dressing as described above may be formed from a first layer 2 and a second layer 3 of a material such as polyester (e.g. Stratex™) separated by an absorptive layer 4 formed from a mix of absorptive substrates. The first layer 2 and second layer 3 are welded together with spot welds at la, 2b, 3c and 4d. The resulting dressing has cavities 5 over the wound 6 and patient's skin 7. As shown in FIGS. 8 and 9, a covering layer 1 may be applied over the segment 2 to assist in protecting the wound 6.

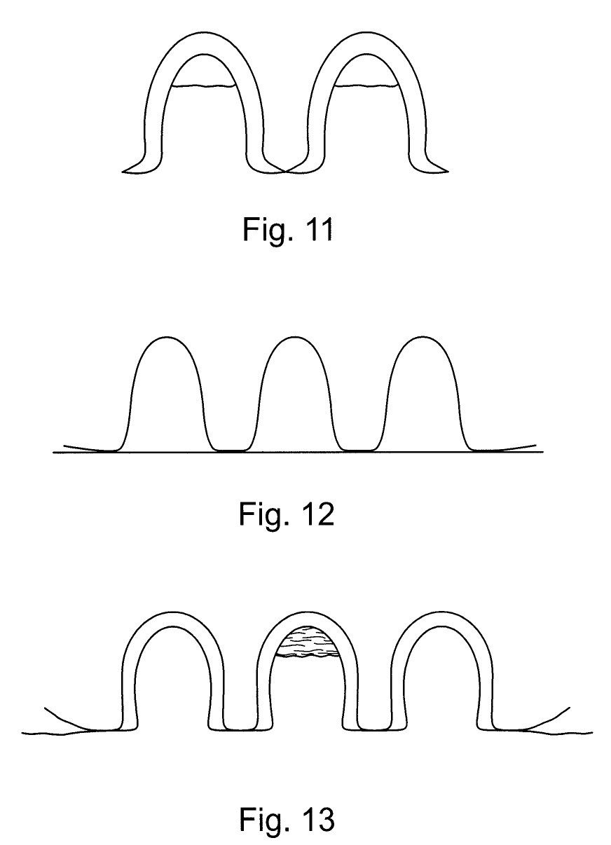

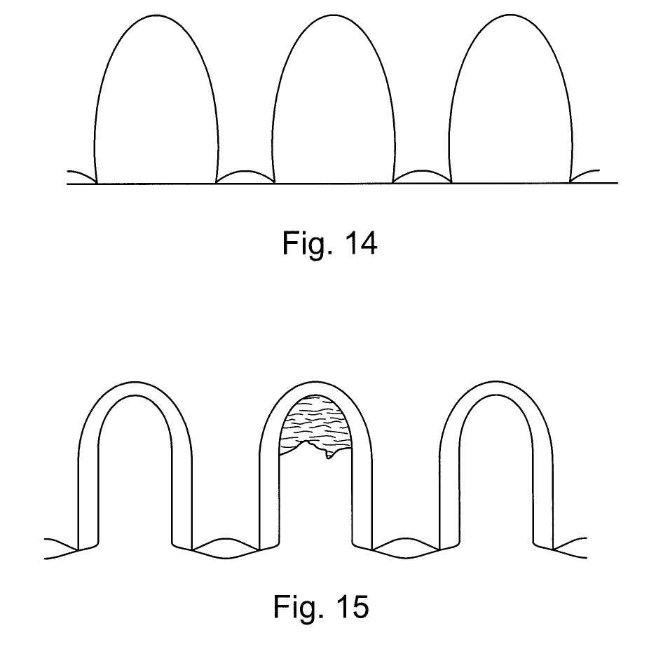

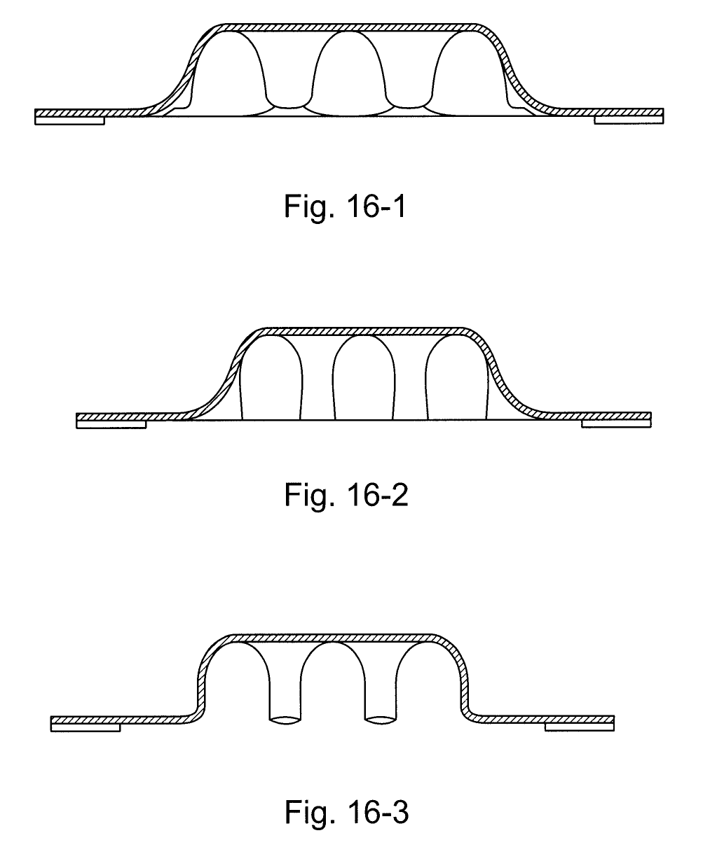

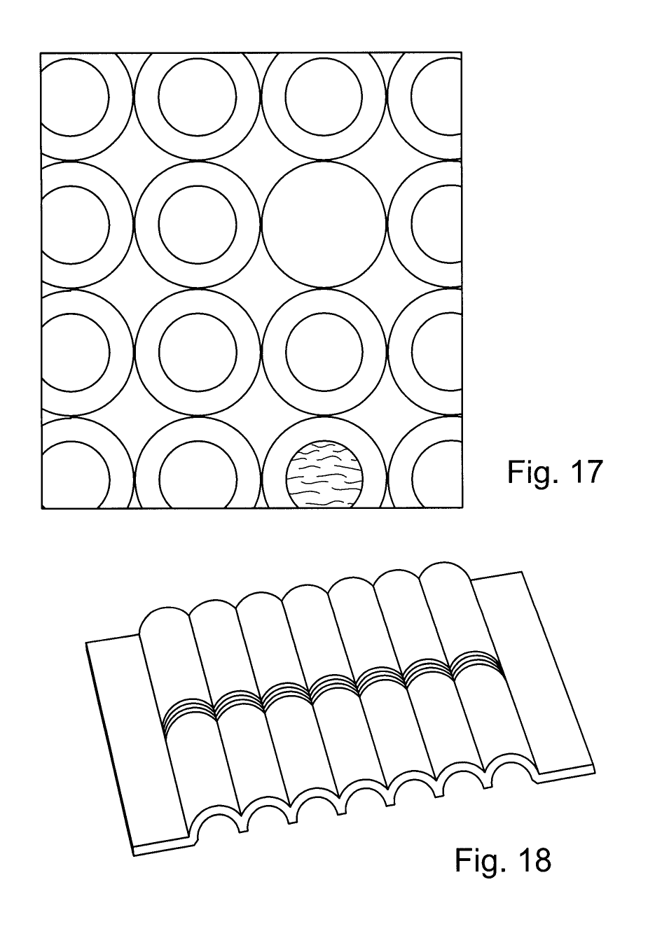

Referring to FIGS. 10 to 16, the cavities may also permit the introduction of medication between the segments of the wound dressing and the wound. FIGS. 10 to 16 show variations in the pattern and spacing of the protrusions. FIG. 17 shows a bottom view of a sheet of wound dressing, with the cavities and medications shown therein. FIG. 18 shows a perspective view of a wound dressing, with channels along the protrusions for flexibility.

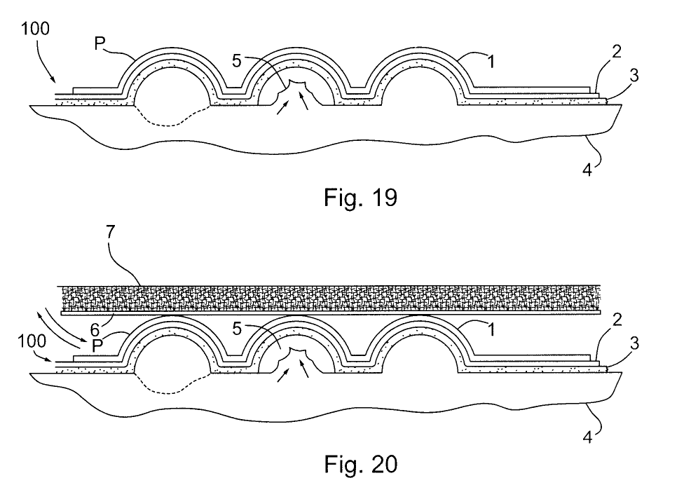

According to an embodiment as shown in FIGS. 19, segments of wound dressing 100 may include a breathable external layer 1, a medicinal layer 2 and an absorptive layer 3, with absorptive layer 3 lying closest to the patient's skin 4. Referring to FIG. 20, the dressing 100 may be covered by a second external layer 6 for use in providing venting with a brace or cast 7.

Layers 1, 2 and 3 may form a protruded layer, wherein each protrusion may range from 0.5 to 50 mm in height and 0.5 to 40 mm in diameter, depending on the size of the user's wound. Each protrusion may store additional medication in the cavity defined therein. According to an embodiment, wound dressing 100 may be used for the treatment of a user's wound, wherein each protrusion may allow room for the growth of new user skin 5.

External layer 1 may be constructed of a breathable material suitable for ventilation. Medicinal layer 2 may be constructed or coated with activated or absorbent carbon, silver, hydrocolloid, honey, calcium alginate, polyurethane foam, gel foam, biocomposite such as collagen, special medicinal ingredients or any other suitable material known to a skilled person for the treatment of user's wound. Absorptive layer 3 may be constructed of an absorptive material such as Stratex™, cotton, calcium alginate, silver, hydrocellular antimicrobial foam, polyurethane foam or any other suitable material known to a skilled person.

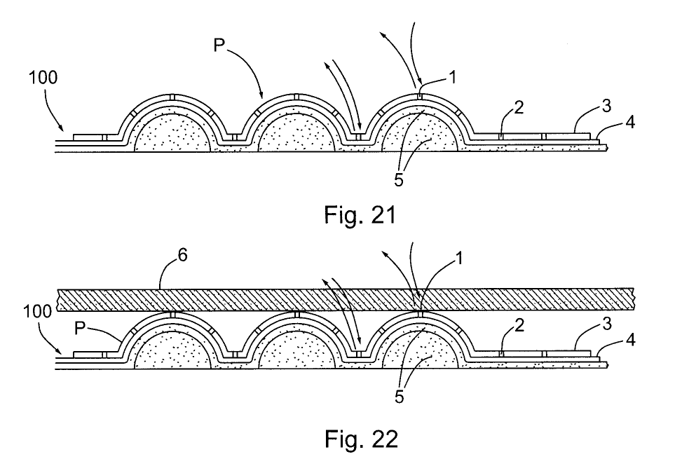

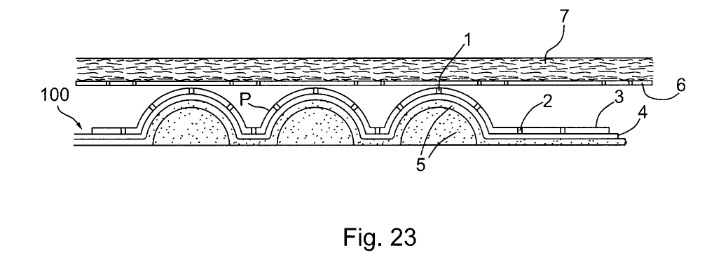

According to an embodiment as shown in FIGS. 21-23, segments of wound dressing 100 may include a hard external layer 3, a medicinal layer 4 and an absorptive layer 5, with absorptive layer 5 lying closest to the patient's skin. External layer 3 may further include holes 1 and 2 to provide ventilation. A hard brace or cast 6 may directly overlay external layer 3, or, as shown in FIG. 23, a second external layer 7 may be provided to produce an even external surface. External layer 3 (and 7, if present) are harder than layers 4 and 5 and may provide protection to the wound from external blows and contact.

External layer 3 may be constructed of thermoplastic such as EVA, Plastazote™ or any other suitable material known to a skilled person. According to an embodiment, external layer 3 may be composed of at least two layers, wherein the internal layer is softer than the external layer and the external layer is harder than the internal layer. Internal layer 5 may be constructed of an absorptive material such as Stratex™, cotton, polyurethane foam, foam, hydrocolloid agent, silver or any other suitable material known to a skilled person. Middle layer 4 may be constructed or coated with activated or absorbent carbon, silver, hydrocolloid, honey, calcium alginate, polyurethane foam, gel foam, biocomposite such as collagen, special medicinal ingredients or any other suitable material known to a skilled person for the treatment of user's wound. Layer 7 may be constructed of thermoformable EVA, Plastazote™ or any other suitable material known to a skilled person.

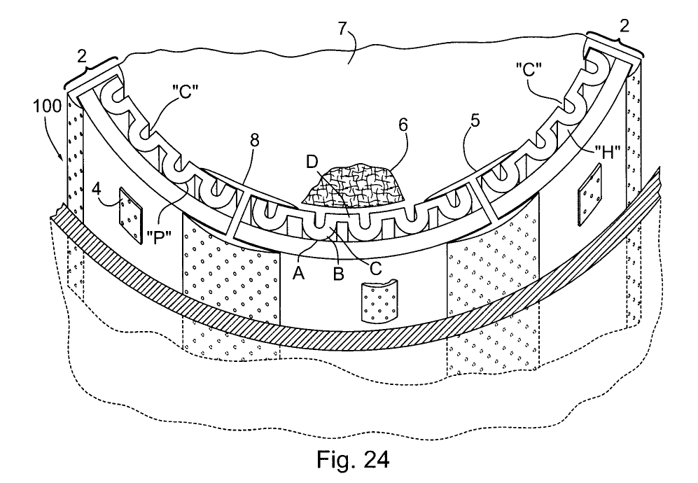

According to an embodiment as shown in FIG. 24, wound dressing 100 is applied to a wound 6 in a patient's skin 7. Dressing 100 may be formed of segments, each segment separated by a spacer, shown as “H” channels 5 and 8, and “U” channels 2. Within each segment there may be a first layer A, a second layer B, a third layer C and a fourth layer D, combining to form a protruded layer with cavities “C” and holes “H”. The protruded layer is covered by a perforated layer 3 to form the segment.

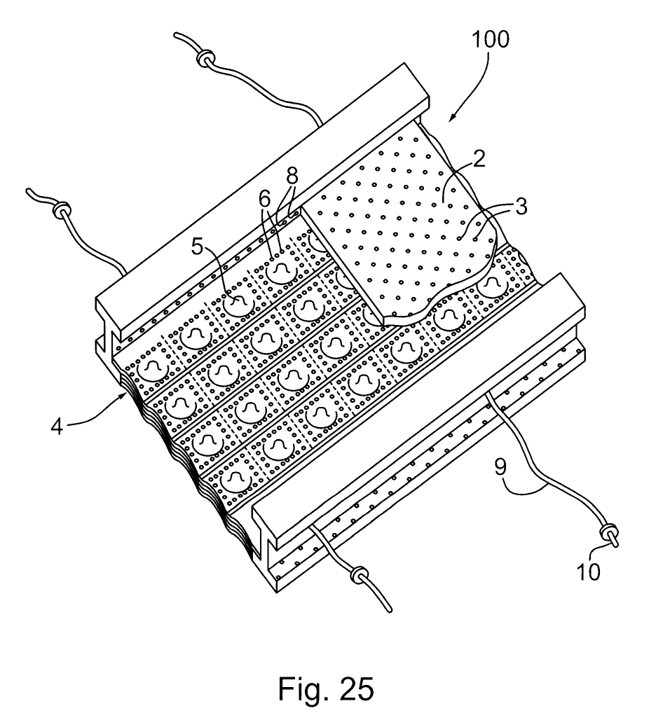

The protruded layer may allow for ventilation, exudate absorption, less frequent changes of the wound dressing 100, and protection of the patient's skin 7 from bacteria. Wound dressing 100 may be attached to an internal wall 1 of an immobilizing orthopedic brace by soft fasteners 4, such as Velcro™, or random and invisible ultrasound spot welds. According to an embodiment as shown in FIG. 25, elastic cords 9 with stoppers 10 at each end of elastic cords 9 may run through perforations on “H” channels 5 and 8, wherein elastic cords 9 may be stretched and anchored at desired area on internal wall 1.

First layer A and second layer B may be perforated or porous, and first layer A may be harder than second layer B to withstand external pressure and prevent harm to wound 6. First layer A may be constructed of a perforated, hard EVA or Plastazote™. Second layer B may be constructed of a breathable polyurethane. Third layer C may be constructed or coated with activated or absorbent carbon, silver, hydrocolloid, honey, calcium alginate, polyurethane foam, gel foam, biocomposites such as collagen, special medicinal ingredients or any other suitable material known to a skilled person for the treatment of user's wound. Fourth layer D may be constructed of a hydrophilic material such as Stratex™, cotton or any other suitable material known to a skilled person.

As an alternative spacer to the “H” channels described above may be fabric spacers, such as XD-Spacer Fabrics available from Baltex (UK). Fabric spacers may be lighter than EVA layers, and may remove the need for thermoforming layers and including perforations as with EVA layers. Fabric spacers may also be substituted for foam cushions and pads inside walking braces. Treatment with silver ions or similar processes may be used to limit bacterial growth and/or speed healing. Another alternative is to replace alternating segments with fabric spacers while retainer the “H” channels between the segments and the fabric spacers.

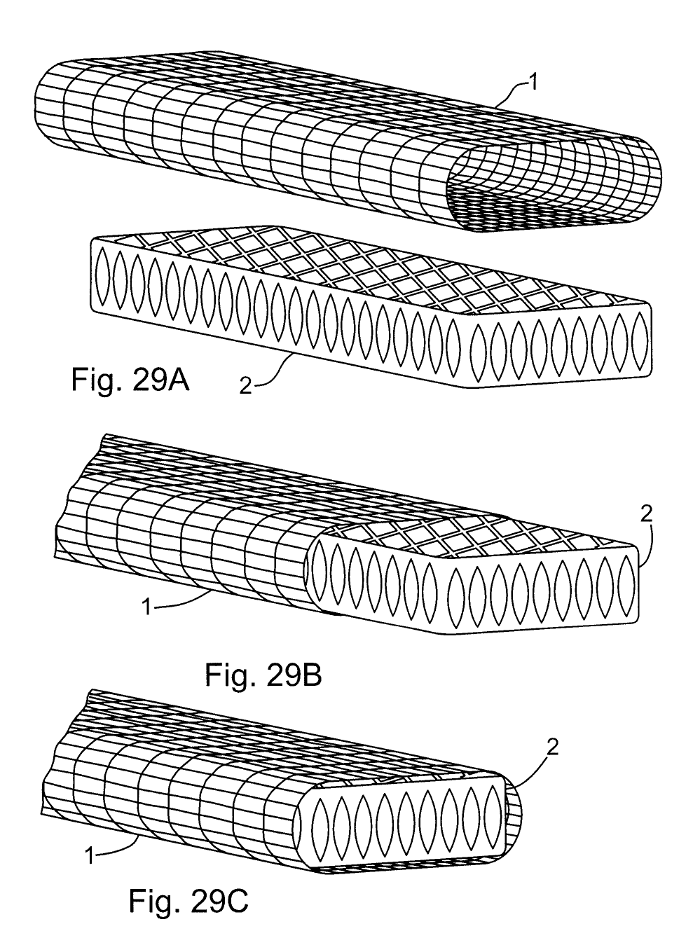

According to FIG. 29A, tubinette 1 is shown as an elongated tube which is expandable in the longitudinal direction. The tubinette may be made of cotton or a similar expandable, sterile material. A fabric spacer 2 is also shown, which may be made of any fabric that is capable of withstanding elongation, particularly lateral elongation. The fabric spacer 2 is of variable thickness, depending on the intended body part for treatment, although a range of 4 mm to 20 mm may be most commonly used. The fabric spacer 2 is dimensioned to fit within tubinette 1, as shown in FIG. 29B, with a fully assembled spacer shown in FIG. 29C.

The tubinette 1 may keep the spacer 2 from sliding under compression when the wound dressing is applied to the wound and placed under pressure. Additionally, tubinette 1 may prevent any filaments or pieces of spacer 2 from coming into contact with the wound.

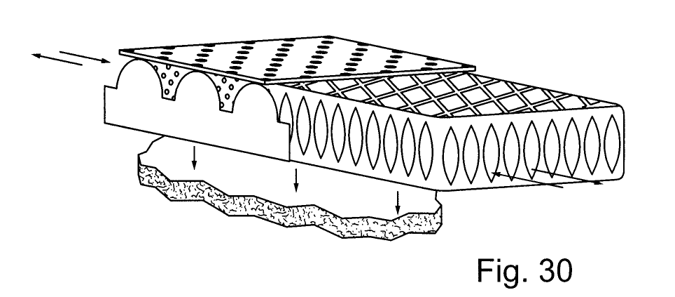

FIG. 30 shows a tubinette encased fabric spacer in position relative to a segment of the wound dressing. As shown, the spacer is equal in size to both layers of the segment, however, the spacer may be thicker or thinner, if desired. The spacer enables ventilation of the wound in combination with the protrusions and perforations of the segment layers.

The overall shape of spacer 2 may be rectangular, square, oval or circular, and may be longitudinally planar, or curved or arched to assist with placement over certain body parts, such as the bicep, or fingers.

According to an embodiment, an alternative to tubinette 1 may be a surgical net, such as Surgilast, which enables expansion in four directions. Four direction expansion may be more effective in wrapping around complex body geometries, such as the ankles or elbows.

The present invention may be embodied in other specific forms without departing from the spirit or essential characteristics thereof. Certain adaptations and modifications of the invention will be obvious to those skilled in the art. Therefore, the presently discussed embodiments are considered to be illustrative and not restrictive, the scope of the invention being indicated by the appended claims rather than the foregoing description and all changes which come within the meaning and range of equivalency of the claims are therefore intended to be embraced therein.

What is claimed is:

1. A wound dressing comprising:
a plurality of individual segments, each segment connected to at least one other segment by an elastic connector, each segment comprising at least two perforated layers, wherein at least one layer comprises a plurality of protrusions; and
a plurality of spacing elements, each spacing element disposed between adjacent segments and retained by either the adjacent segments or the elastic connector.

2. The wound dressing of claim 1, wherein each segment is similar in size and shape.

3. The wound dressing of claim 1, wherein the at least two layers are attached by soft fasteners.

4. The wound dressing of claim 1, wherein each segment is additionally connected to a consecutive segment by the spacing element.

5. The wound dressing of claim 4, wherein the spacing elements are connected to each segment with soft fasteners.

6. The wound dressing of claim 1, wherein the spacing elements comprise a hypoallergenic material.

7. The wound dressing of claim 1, wherein the at least two layers are enclosed by a removable envelope.

8. The wound dressing of claim 1, wherein each segment is enclosed by a removable envelope.

9. The wound dressing of claim 1, wherein spacing elements further comprise at least one elastic thread with stoppers at each end of the at least one elastic thread that runs transversally through walls of the pacing elements and anchors at a desired area of an orthopedic device.

10. The wound dressing of claim 1, wherein the spacing elements are porous.

11. The wound dressing of claim 1, wherein the at least two layers includes an inner layer comprising an absorptive material.

12. The wound dressing of claim 1, wherein the at least two layers includes an exterior layer comprising a breathable material.

13. The wound dressing of claim 1, wherein one of the at least two layers includes a medicinal substance.

14. The wound dressing of claim 1, wherein the at least two layers includes an exterior layer having a greater hardness than an inner layer.

15. The wound dressing of claim 1, wherein at least one layer of the at least two layers comprises a hypoallergenic material.

16. The wound dressing of claim 1, wherein at least one layer is thermoformable and comprises a thermoplastic material.

17. The wound dressing of claim 1, wherein the spacing elements are deformable along at least one axis planar with the expandable connector.

18. The wound dressing of claim 1, wherein the spacing elements are similar in size and shape.

19. The wound dressing of claim 1, wherein the spacing elements are similar in thickness to the at least two layers.

20. The wound dressing of claim 1, wherein the elastic connector is an elastic net formed from Surgilast™.
